SEQUENCE LISTING (17 sequences)

SEQ ID NO: 1 - DNA, 26 bp, Artificial sequence
  Synthetic nucleic acid sequence
ctagaagctttgtgaaacagctgccc

SEQ ID NO: 2 - DNA, 27 bp, Artificial sequence
  Synthetic nucleic acid sequence
cgctagtctagatgtctctcaaatttc

SEQ ID NO: 3 - DNA, 52 bp, Homo Sapiens
  misc_feature (27)..(27): n at this position can be a “A” or a “G”
gctcgaattgagaaactagttttttcnataactggctattcgatcagcattc

SEQ ID NO: 4 - DNA, 52 bp, Homo Sapiens
  misc_feature (27)..(27): n at this position can be a “C” or a “G”
acagcccatcacatgctcaatacattnaagtcaaagaaacctcatagcacag

SEQ ID NO: 5 - DNA, 52 bp, Homo Sapiens
  misc_feature (27)..(27): n at this position can be a “A” or a “C”
agaaacctcatagcacagctatgtccnctgttcccctcaacaacccacacac

SEQ ID NO: 6 - DNA, 52 bp, Homo Sapiens
  misc_feature (27)..(27): n at this position can be a “C” or a “T”
tacaacttctaactgatagagtaatgntgacataacagtttgtgactctggg

SEQ ID NO: 7 - DNA, 52 bp, Homo Sapiens
  misc_feature (27)..(27): n at this position can be a “A” or a “G”
tcctctccttgtgctaggccaggctgngaggtctacaggagatcagggaggc

SEQ ID NO: 8 - DNA, 52 bp, Homo Sapiens
  misc_feature (27)..(27): n at this position can be a “C” or a “T”
ggggtctgaaaggaagagtcagaaaantcaagcactttgcatctctcatggg

SEQ ID NO: 9 - DNA, 52 bp, Homo Sapiens
  misc_feature (27)..(27): n at this position can be a “G” or a “T”
gcatggaggtgggggtcgtgatctgcnccctgggccggagttactcactggc

SEQ ID NO: 10 - DNA, 52 bp, Homo Sapiens
  misc_feature (27)..(27): n at this position can be a “A” or a “G”
ttttttcaataactggctattcgatcngcattctataatacacttaacatct

SEQ ID NO: 11 - DNA, 52 bp, Homo Sapiens
  misc_feature (27)..(27): n at this position can be a “A” or a “G”
ggaccttctcctgacctgtgattactnggatcaggtccccatcactacaatc

SEQ ID NO: 12 - DNA, 52 bp, Homo Sapiens
  misc_feature (27)..(27): n at this position can be a “A” or a “G”
caggaccagagggagggcgatattctngtgttggtcccattttcctcctctc

SEQ ID NO: 13 - DNA, 52 bp, Homo Sapiens
  misc_feature (27)..(27): n at this position can be a “C” or a “T”
atccccaccaccctctcactcctgaantgtggctctagcactgttccatgac

SEQ ID NO: 14 - RNA, 22 nt, Homo Sapiens
ucagugcacuacagaacuuugu

SEQ ID NO: 15 - RNA, 22 nt, Homo Sapiens
ucagugcaucacagaacuuugu

SEQ ID NO: 16 - RNA, 22 nt, Homo Sapiens
ucagugcaugacagaacuuggg

SEQ ID NO: 17 - DNA, 4634 bp, Homo Sapiens
tgttttaggtggagaaatgacacactctgactcatagtagcaggaccactatagagagaacactcatgtagcaggtcatggaacagtgctagagccacaattcaggagtgagagggtggtggggattaaggggagaagagggcctgagggatgagagggacggagggaagggctggaggagcaggaggtgaggaaaaggagcagaggaaagaattccaaagcagcggaactcttaggtttaaacacattgttttatagattttattacatccatctacagagcctcgctgggtgttctttgcagttggcctttaatatcttatgtgggtctgcctagaaactaattgttttttatgttaatcaggtttaaaaaatactaagtattcctaaaaaatatacactccactcacatgtggatacttcctaaaaacaggcagtgcatgagcactagtgaggggcattgtgactgcactgaacacttacaactgtgaggtgaataaagtttgtgctggctcctggttgcaacatatagtaacatagtgtggtactttgtcttgaggagatgtcctggactcacacggaaacttagggctacggaatgaaggtaaatttaaaataaaacaagcgggagtcacagatacattgtctgggaaagtgaaacttaagagctttgtgagtcctgttgtaaggcttttagatgcatttatataccaacgggccaaagtcacattttttacctattagattcctgatcattcaggggttaccaagattatgctacccactatagttaataaacaaaaagcaaactggtctctattctatctcatgcactcaggcacaacttttccagatttaagggggaaaaaaaaccctgtctttacacctacaatcccagggcgagctcactctctggcaccaagctccgtggggtgatttttcttctagaagagtacaggaggacaggcaaggagtgggaggcagggagtccagttcagggacagggattccgggatgaaaagtgaagggagagggacagggaccttgccgagggtttctccctggtttctcagacagctcctgggccaagactcagggagacactgagacagaacgcttggcacaagagtagcggggtcagggcgaagtcccagggcctcaagcgtggctctcagggtctcaggccccacaggcggtgtatggattggggaggccccgcgttggggattctctcctccttctcctaacctgtgtcgggtccttcttcctggatactcaccgggcggccccagttctcactcccattaggtgacaggtttttagagaagccaatcagcgtcgccgcggtcctggttctaaagtcctcgctcacccacccggactcattctccccagacgccaaggatggtggtcatggcaccccgaaccctcttcctgctactctcgggggccctgaccctgaccgagacctgggcgggtgagtgcggggtcaggagggaaacggcccctgcgcggaggagggaggggccggcccggcgggggcgcaggacccggcagccgcgccgggaggagggtcgggcgggtctcaacccctcctcgcccccaggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccagagtattgggaagaggagacacggaacaccaaggcccacgcacagactgacagaatgaacctgcagaccctgcgcggctactacaaccagagcgaggccagtgagtaaccccggcccagggcgcagatcacgaccccccacctccatgccccacggacgccccgggtactcccgagtctccgggtctgggatccaccccgaggccgcgggacccgcccagaccctctacctgggagaaccccaggcgcctttaccaaaatccctgcgggtgggtccgggcgagggcgaggctcggtgggcggggctgaccgaaggggtggggccaggttctcacaccctccagtggatgattggctgcgacctggggtccgacggtcgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcgggtaccaggggcagtggggcgcctccctgatctcctgtagacctcccagcctggcctagcacaaggagaggaggaaaatgggaccaacaccagaatatcgccctccctctggtcctgagggagaggaatcctcctgggtttccagatcctgtacaagagagtgattctgagggcccgtcctgctctctgggacaattaagggatgaagtctctgagggagtggaggggaagacaatccctggaggactgatcaggggttccctttgaccccacagcagccttggcaccaggacttttcccctcaggccttgttctctgcctcacactcaatgtgtgtgggagtctgactccagctcctctgagtcccttggcctccactcaggtcagaaccagaggtccctgctcccccgctcagagactagaactttccaaggaataggagattatcccaggtgcccgtgtccaggctggtgtctgggttctgtgctcccttccccaccccaggtatctggttcattcttaggatggtcacatccaggtgctgctggagtgtcccatgagagatgcaaagtgcttgagttttctgactcttcctttcagacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggagtaaggagggagatggaggcatcatgtctgttagggaaagcaggagcctctctgaagacctttaacagggtcggtggtgaggcctgggggtcagagaccctcaccttcacctcctttcccagagcagtcttccctgcccaccatccccatcatgggtatcgttgctggcctggttgtccttgcagctgtagtcactggagctgcggtcgctgctgtgctgtggaggaagaagagctcaggtaaggaaggggtgacaagtggggtctgagttttcttgtcccactgggggtttcaagccccaggtagaagtgtgccctgcctggttactgggaagcaccatccacactcatgggcctacccagcctgggccctgtgtgccagcaccttctcttttgtaaagcacctgtgacaatgaaggacagatttatcaccttgatgattgtagtgatggggacctgatcctagtaatcacaggtcaggggaaggtccctggctaaggacagaccttaggagggcagttggtcgaggacccacatctgctttccttgtttttcctgatcccgccctgagtctgcagtcacacatttctggaaacttctcgagggtccaagattaggaggttcctctaggacctcatggccctgccacctttctggcctctcacaggacgttttcttcccacagattgaaaaggagggagctactctcaggctgcaagtaagtatgaaggaggctgatccctgagatccttgggatcttgtgtttgggagcccatgggggagctcacccaccccacaattcctcctctggccacatctcctgtggtctctgaccaggtgctgtttttgttctactctaggcagtgacagtgcccagggctctaatgtgtctctcacggcttgtaaatgtgacaccccggggggcctgatgtgtgtgggttgttgaggggaacagtggacatagctgtgctatgaggtttctttgacttgaatgtattgagcatgtgatgggctgtttaaagtgtcacccctcactgtgactgatatgaatttgttcatgaatatttttctgtagtgtgaaacagctgccctgtgtgggactgagtggcaagatttgttcatgccttccctttgtgacttcaagaaccctgacttctctttctgcagagaccagcccaaccctgtgtccaccatgaccctcttcctcatgctgaactgcattccttccccaatcacctttcctgttccagaaaaggggctgggatgtctccgtctctgtctcaaatttgtggtgcactgagctataacttacttctgtattaaaattagaatctgagtataaatttactttttcaaattatttccaagagagattgatgggt

(19) United States
(12) Patent Application Publication
(10) Pub. No.: US 2013/0225670 A1
(43) Pub. Date: Aug. 29, 2013

(54) Methods of Treating Asthma Using Statins

(71) Applicant: Children's Hospital & Research Center Oakland, (US)

(72) Inventors: Marisa Lin Wong Medina, Hercules, CA (US); Ronald M. Krauss, Berkeley, CA (US)

(73) Assignee: Children's Hospital & Research Center Oakland, Oakland, CA (US)

(21) Appl. No.: 13/776,424

(22) Filed: Feb. 25, 2013

Related U.S. Application Data
(60) Provisional application No. 61/605,063, filed on Feb. 29, 2012.

(51) Int. Cl. C12Q 1/68; A61K 31/366

(52) U.S. Cl. CPC C12Q 1/6883; A61K 31/366
USPC 514/460; 506/2

(57) ABSTRACT

The present disclosure generally provides methods for assessing a subject's responsiveness to a statin therapy, and selection of a statin dose based upon such methods. The disclosure further provides methods for treating asthma by administering a statin therapy.

### CROSS-REFERENCE TO RELATED APPLICATIONS

This application claims priority benefit of U.S. Provisional Application Ser. No. 61/605,063, filed on Feb. 29, 2012, the disclosure of which application is herein incorporated by reference in its entirety.

### STATEMENT REGARDING FEDERALLY SPONSORED RESEARCH

This invention was made with government support under NIH grant U01-HL067957. The government has certain rights in this invention.

### BACKGROUND

Asthma is a disorder resulting from a combination of genetic and environmental factors that cause the airways of the lungs to swell and narrow, leading to symptoms including wheezing, shortness of breath, chest tightness, and coughing. Most patients who suffer from asthma have “attacks” that are separated by symptom-free periods. Asthma attacks can last anywhere from a few minutes up to several days, and can become dangerous if airflow to the lungs is severely restricted.

Treatment of asthma generally involves administering control drugs that prevent attacks, and using quick-relief drugs to alleviate symptoms during attacks. Control drugs that are used to prevent asthma attacks generally include anti-inflammatory medications, such as inhaled corticosteroids. There is a need to understand the relationship between the genetic factors that give rise to asthma and the effects of anti-inflammatory compounds, such as statins, in order to more effectively use such compounds in management of the disease.

### SUMMARY

The present disclosure generally provides methods for assessing a subject's responsiveness to a statin therapy, and selection of a statin dose based upon such methods. The disclosure further provides methods for treating asthma by administering a statin therapy.

In some embodiments, the present disclosure provides methods for determining a likelihood of a beneficial response to statin therapy in an asthma patient, the methods including genotyping the single nucleotide polymorphism (SNP) rs1063320, or a surrogate SNP thereof, in a sample of genetic material from the patient, wherein the genotyping determines if the nucleotide of the SNP rs1063320 of one or both alleles of the patient's HLA-G gene is a guanine or a cytosine; wherein a cytosine at position +3142 of one or both alleles is negatively associated with the likelihood that the patient will have a beneficial response to statin treatment; and wherein a guanine at position +3142 of one or both alleles is positively associated with the likelihood that the patient will have a beneficial response to statin treatment.

In some embodiments, the subject methods include generating a report indicating the genotype at position +3142 of one or both of the HLA-G alleles in the asthma patient. In some embodiments, the report includes an indication of the likelihood of a beneficial response to statin therapy in the asthma patient based on the genotype at position +3142 of one or both of the patient's HLA-G alleles.

In some embodiments, the present disclosure provides methods for determining a statin dose to administer to an asthma patient, the methods including genotyping the single nucleotide polymorphism (SNP) rs1063320, or a surrogate SNP thereof, in a sample of genetic material from the patient, wherein said genotyping determines if the nucleotide of the SNP rs1063320 of one or both alleles of the patient's HLA-G gene is a guanine or a cytosine; wherein a cytosine at position +3142 of one or both alleles indicates a higher dose of statin than is indicated for a patient with a guanine at position +3142 of both alleles.

In some embodiments, the subject methods include genotyping the single nucleotide polymorphism (SNP) rs1063320, or a surrogate SNP thereof, in a sample of genetic material from the patient, wherein the genotyping determines if the nucleotide of the SNP rs1063320 of one or both alleles of the patient's HLA-G gene is a guanine or a cytosine; wherein if a cytosine is present at position +3142 of one or both alleles, an initial recommended statin dose is administered to the patient and the statin dose is optionally increased until a reduction in asthma symptoms is observed; and if a guanine is present at position +3142 of both alleles, an initial recommended statin dose is administered to the patient and the statin dose is optionally decreased so long as adequate control of symptoms is maintained.

In some embodiments, the subject methods include generating a report with information to guide a clinician's recommendation with respect to statin dose for the patient based on the nucleotide at position +3142 of one or both alleles of the patient's HLA-G gene.

In some embodiments, the present disclosure provides methods of treating a patient having asthma, the methods including determining a statin dose as described above, and administering the statin dose to the patient.

In some embodiments, the present disclosure provides methods of treating a patient having asthma, the methods including administering a statin to the patient in an amount effective to treat asthmatic symptoms in the patient, wherein the patient has a guanine at position +3142 of both HLA-G alleles, or has a cytosine at position +3142 of both HLA-G alleles, or has a guanine at position +3142 of one HLA-G allele and has a cytosine at position +3142 of the other HLA-G allele.

Other features, embodiments and various advantages of the invention will be readily apparent to the ordinarily skilled artisan upon reading the disclosure herein.

### BRIEF DESCRIPTION OF THE DRAWINGS

FIG. 1 is a bar graph that shows the change in mir-148a, 148b and 152 in lymphoblast cell lines (LCLs) (first series) and a hepatocyte cell line (second series) after 24 hours of 2.0 μM simvastatin treatment. Values are expressed as fold change from sham treated cells. An asterisk indicates up-regulation by statin treatment, p<0.05.

FIG. 2 is a series of graphs showing the correlation between HLA-G mRNA and mir-148a, 148b, and 152 in lymphoblast cell lines split by rs1063320 genotype. GG, n=11. CC or CG, n=15. Both HLA-G and mRNA levels are expressed as quantile normalized values.

FIG. 3 is a series of bar graphs showing HLA-G mRNA and protein levels measured in lymphoblast cell lines split by rs1063320 genotype.

FIG. 4 is an example of a human HLA-G sequence. The ATG start codon is identified with a box, and the nucleotide at position +3142 is identified with a box as well.

### DEFINITIONS

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a stem cell” includes a plurality of such stem cells and reference to “the stem cell marker” includes reference to one or more stem cell markers and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As used herein, the term “treating” in reference to a disorder means a reduction in severity of one or more symptoms associated with a particular condition. Therefore, treating a condition does not necessarily mean a reduction in severity of all symptoms associated with a condition and does not necessarily mean a complete reduction in the severity of one or more symptoms associated with a condition. “Treatment”, as used in this context, covers any treatment of a symptomatic condition in a human, and includes: (a) diagnosing and then preventing the condition from occurring in a subject who may be predisposed but has not yet been diagnosed as having it; (b) inhibiting the condition, i.e., arresting its development; and (c) relieving the condition, i.e., causing regression of the condition. Similarly, the term “preventing” means prevention of the occurrence or onset of one or more symptoms associated with a particular condition and does not necessarily mean the complete prevention of the condition.

The terms “individual,” “subject,” and “patient,” used interchangeably herein, generally refer to a human subject, such as, e.g., a human subject undergoing treatment for asthma.

The term “dose” as used herein generally refers to an amount of a compound that is administered to a subject in a single administration.

The term “dosage regimen” as used herein generally refers to a dose and frequency of administration of the dose (e.g., 20 mg/day).

As used herein, the term “biological sample” or “sample” refers to a sample of tissue or fluid isolated from a subject, which, depending on the context in which the term is used, generally refers to a sample that contains genetic material from a subject and that is suitable for HLA-G genotyping analysis. Non-limiting examples of sample types include blood, plasma, serum, saliva, buccal swabs, and the like. Where a sample includes cells for analysis, the sample can be provided as in vitro cell culture constituents, including but not limited to conditioned media resulting from the growth of cells and tissues in cell culture medium.

As used herein, the term “genotyping” means analyzing a sample that includes a target nucleic acid to determine the identity of a nucleotide present at a position of interest in the target nucleic acid.

As used herein, the term “target nucleic acid region” or “target nucleic acid” or “target molecules” refers to a nucleic acid molecule with a “target sequence” to be detected (e.g., by amplification). The target nucleic acid in the present disclosure is usually an HLA-G gene sequence. Where detection is by amplification, other sequences in addition to the target sequence may or may not be amplified with the target sequence.

The term “target sequence” or “target nucleic acid sequence” refers to the particular nucleotide sequence of the target nucleic acid to be detected (e.g., through amplification). The target sequence may include a probe-hybridizing region contained within the target molecule with which a probe will form a stable hybrid under desired conditions. The “target sequence” may also include the complexing sequences to which the oligonucleotide primers complex and are extended using the target sequence as a template. Where the target nucleic acid is originally single-stranded, the term “target sequence” also refers to the sequence complementary to the “target sequence” as present in the target nucleic acid, e.g., present in an amplification product generated from an HLA-G nucleotide sequence. Moreover, where sequences of a “target sequence” are provided herein, it is understood that the sequence may be either DNA or RNA. Thus, where a DNA sequence is provided, the RNA sequence is also contemplated and is readily provided by substituting “T” of the DNA sequence with “U” to provide the RNA sequence.

The term “primer” or “oligonucleotide primer” as used herein, refers to an oligonucleotide that acts to initiate synthesis of a complementary nucleic acid strand when placed under conditions in which synthesis of a primer extension product is induced, e.g., in the presence of nucleotides and a polymerization-inducing agent such as a DNA or RNA polymerase and at suitable temperature, pH, metal concentration, and salt concentration. Primers are generally of a length compatible with its use in synthesis of primer extension products, and are usually in the range of between 8 to 100 nucleotides in length, such as 10 to 75, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. Typical primers can be in the range of between 10-50 nucleotides long, such as 15-45, 18-40, 20-30, 21-25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length.

Primers are usually single-stranded for maximum efficiency in amplification, but may alternatively be double-stranded. If double-stranded, the primer is usually first treated to separate its strands before being used to prepare extension products. This denaturation step is typically affected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

A “primer pair” as used herein refers to first and second primers having nucleic acid sequences suitable for nucleic acid-based amplification of a target nucleic acid. Such primer pairs generally include a first primer having a sequence that is the same or similar to that of a first portion of a target nucleic acid, and a second primer having a sequence that is complementary to a second portion of a target nucleic acid to provide for amplification of the target nucleic acid or a fragment thereof. Reference to “first” and “second” primers herein is arbitrary, unless specifically indicated otherwise. For example, the first primer can be designed as a “forward primer” (which initiates nucleic acid synthesis from a 5′ end of the target nucleic acid) or as a “reverse primer” (which initiates nucleic acid synthesis from a 5′ end of the extension product produced from synthesis initiated from the forward primer). Likewise, the second primer can be designed as a forward primer or a reverse primer.

As used herein, the term “probe” or “oligonucleotide probe” refers to a structure comprised of a polynucleotide, as defined above, that contains a nucleic acid sequence complementary to a nucleic acid sequence present in the target nucleic acid analyte (e.g., a nucleic acid amplification product). The polynucleotide regions of probes may be composed of DNA, and/or RNA, and/or synthetic nucleotide analogs. Probes are generally of a length compatible with its use in specific detection of all or a portion of a target sequence of a target nucleic acid, and are usually are in the range of between 8 to 100 nucleotides in length, such as 8 to 75, 10 to 74, 12 to 72, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. The typical probe is in the range of between 10-50 nucleotides long, such as 15-45, 18-40, 20-30, 21-28, 22-25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length.

Probes contemplated herein include probes that include a detectable label. For example, when an “oligonucleotide probe” is to be used in a 5′ nuclease assay, such as the TaqMan™ assay, the probe includes at least one fluorescer and at least one quencher, where the probe is digested by a 5′ endonuclease activity so as to provide for a detectable signal as a result of specific binding of probe to any amplified target oligonucleotide sequences. In this context, the oligonucleotide probe will have a sufficient number of phosphodiester linkages adjacent to its 5′ end so that the 5′ to 3′ nuclease activity employed can efficiently degrade the bound probe to separate the fluorescers and quenchers.

As used herein, the terms “label” and “detectable label” refer to a molecule capable of detection, including, but not limited to, radioactive isotopes, fluorescers, chemiluminescers, chromophores, enzymes, enzyme substrates, enzyme cofactors, enzyme inhibitors, chromophores, dyes, metal ions, metal sols, ligands (e.g., biotin, avidin, strepavidin or haptens) and the like. The term “fluorescer” refers to a substance or a portion thereof which is capable of exhibiting fluorescence in the detectable range.

The terms “hybridize” and “hybridization” refer to the formation of complexes between nucleotide sequences that are sufficiently complementary to form complexes via Watson-Crick base pairing. Where a primer “hybridizes” with target (template), such complexes (or hybrids) are sufficiently stable to serve the priming function required by, e.g., the DNA polymerase to initiate DNA synthesis.

As used herein, the term “binding pair” refers to first and second molecules that specifically bind to each other, such as complementary polynucleotide pairs capable of forming nucleic acid duplexes. “Specific binding” of the first member of the binding pair to the second member of the binding pair in a sample is evidenced by the binding of the first member to the second member, or vice versa, with greater affinity and specificity than to other components in the sample. The binding between the members of the binding pair is typically noncovalent.

By “selectively bind” is meant that the molecule binds preferentially to the target of interest or binds with greater affinity to the target than to other molecules. For example, a DNA molecule will bind to a substantially complementary sequence and not to unrelated sequences.

The term “stringent conditions” refers to conditions under which a primer will hybridize preferentially to, or specifically bind to, its complementary binding partner, and to a lesser extent to, or not at all to, other sequences. Put another way, the term “stringent hybridization conditions” as used herein refers to conditions that are compatible to produce duplexes between complementary binding members, e.g., between probes and complementary targets in a sample, e.g., duplexes of nucleic acid probes, such as DNA probes, and their corresponding nucleic acid targets that are present in the sample.

“Stringent hybridization” and “stringent hybridization wash conditions” in the context of nucleic acid hybridization (e.g., as in array, Southern or Northern hybridizations) are sequence dependent, and are different under different environmental parameters. Stringent hybridization conditions that can be used to identify nucleic acids within the scope of the invention can include, e.g., hybridization in a buffer comprising 50% formamide, 5 SSC, and 1% SDS at 42 C., or hybridization in a buffer comprising 5 SSC and 1% SDS at 65 C, both with a wash of 0.2 SSC and 0.1% SDS at 65 C. Examples of stringent hybridization conditions can also include a hybridization in a buffer of 40% formamide, 1 M NaCl, and 1% SDS at 37 C., and a wash in 1 SSC at 45 C. Alternatively, hybridization to filter-bound DNA in 0.5 M NaHPO4, 7% sodium dodecyl sulfate (SDS), 1 mnM EDTA at 65 C., and washing in 0.1 SSC/0.1% SDS at 68 C. can be employed. Additional stringent hybridization conditions include hybridization at 60° C. or higher and 3 SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions is used to determine whether a nucleic acid is specifically hybridized to a probe. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2 SSC at a temperature of at least about 50° C. or about 55° C. to about 60° C. for about 15 to about 20 minutes; or, the hybridization complex is washed twice with a solution with a salt concentration of about 2 SSC containing 0.1% SDS at room temperature for 15 minutes and then washed twice by 0.1 SSC containing 0.1% SDS at 68° C. for 15 minutes; or, equivalent conditions. Stringent conditions for washing can also be, e.g., 0.2 SSC/0.1% SDS at 42° C. In instances wherein the nucleic acid molecules are deoxyoligonucleotides (“oligos”), stringent conditions can include washing in 6 SSC/0.05% sodium pyrophosphate at 37° C. (for 14-base oligos), 48° C. (for 17-base oligos), 55° C. (for 20-base oligos), and 60° C. (for 23-base oligos). See Sambrook, Ausubel, or Tijssen for detailed descriptions of equivalent hybridization and wash conditions and for reagents and buffers, e.g., SSC buffers and equivalent reagents and conditions.

Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed, as appropriate.

A Tm is the temperature in degrees Celsius at which 50% of a polynucleotide duplex made of complementary strands hydrogen bonded in anti-parallel direction by Watson-Crick base pairing dissociates into single strands under conditions of the experiment. The Tm of a DNA molecule depends on its length and on its base composition. DNA molecules rich in GC base pairs have a higher Tm than those having an abundance of AT base pairs. Separated complementary strands of DNA spontaneously re-associate or anneal to form duplex DNA when the temperature is lowered below the Tm. The highest rate of nucleic acid hybridization occurs approximately 25° C. below the Tm. Tm may be predicted according to a standard formula, such as:

Tm=81.5+16.6 log [X+]+0.41 (% G/C)−0.61 (% F)−600/L

where [X+] is the cation concentration (usually sodium ion, Na+) in mol/L; (% G/C) is the number of G and C residues as a percentage of total residues in the duplex; (% F) is the percent formamide in solution (wt/vol); and L is the number of nucleotides in each strand of the duplex.

The term “assessing” includes any form of measurement, and includes determining if an element is present or not. The terms “determining”, “measuring”, “evaluating”, “assessing” and “assaying” are used interchangeably and includes quantitative and qualitative determinations. Assessing may be relative or absolute. “Assessing the presence of includes determining the amount of something present, and/or determining whether it is present or absent. As used herein, the terms “determining,” “measuring,” and “assessing,” and “assaying” are used interchangeably and include both quantitative and qualitative determinations.

### DETAILED DESCRIPTION

The present disclosure generally provides methods for assessing an asthma patient's responsiveness to a statin therapy, and selection of a statin dose based upon such methods. The disclosure further provides methods for treating asthma by administering a statin therapy.

### Overview

The methods of the present disclosure are based on the discovery that statin treatment up-regulates the expression of three micro-RNAs (mirs) whose expression levels correlate with the expression of genetic factors that lead to asthma, such as HLA-G. Without being held to theory, the three mirs can affect a decrease in asthma symptoms by decreasing HLA-G gene expression. The ability of the mirs to decrease HLA-G gene expression may be dictated, at least in part, by the genotype of a patient's HLA-G gene. As such, in some embodiments the subject methods involve determining a patient's HLA-G genotype and using the genotype information to determine a likelihood that the patient will have a beneficial response to statin therapy for the treatment of asthma. In some embodiments, the subject methods involve selecting a statin dosage regimen to be administered to a patient for the treatment of asthma based on the patient's HLA-G genotype. In some embodiments, the subject methods involve administering the selected statin dosage to the patient for the treatment of asthma.

### Statins

In some embodiments, the subject methods involve selecting an appropriate dose of a statin to administer to a patient for the treatment of asthma. Statins are a broad class of compounds that inhibit the activity of the enzyme 3-hydroxy-3-methylglutaryl coenzyme A (HMG-CoA) reductase. Examples of statins that may be used in connection with the subject methods include, but are not limited to, lovastatin (MEVACOR™; see, U.S. Pat. No. 4,231,938); simvastatin (ZOCOR™; see, U.S. Pat. No. 4,444,784); pravastatin sodium (PRAVACHOL™; see, U.S. Pat. No. 4,346,227); fluvastatin sodium (LESCOL™; see, U.S. Pat. No. 5,354,772); atorvastatin (LIPITOR™; see, U.S. Pat. No. 5,273,995); rosuvastatin (CRESTOR™); and pitatvastatin (LIVALO™). The structural formulas of these and additional HMG-CoA reductase inhibitors are described at page 87 of M. Yalpani, “Cholesterol Lowering Drugs,” Chemistry & Industry, pp. 85-89 (Feb. 5, 1996).

Statins generally vary in potency and have a recommended initial or starting dose based on treatment guidelines for treatment of hypercholesterolemia as discussed in more detail below.

### Subjects

The methods of the present disclosure are suitable for any subject for whom asthma treatment is proposed, or any subject who is undergoing asthma therapy. For example, the subject can be one for whom an asthma therapy is proposed due to diagnosis of one or more types of asthma, or presentation with one or more risk factors for asthma, particularly a subject having a family history of asthma, or a subject suffering from asthma symptoms. Subjects may include those having allergic asthma resulting from inflammation of the airways in response to allergens, exercise-induced asthma that occurs with physical exertion, nighttime asthma, e.g., asthma that occurs during sleeping and/or when lying down, or any other type of asthma or asthma-related symptoms. Subjects may include adults as well as children.

Asthmatic subjects may be identified using any of a variety of different diagnosis techniques for determining whether a subject has asthma or has symptoms related to asthma. For example, subjects may be diagnosed with asthma by conducting a physical examination to determine whether a subject has symptoms such as difficulty breathing, wheezing, a runny nose, swollen nasal passages and/or tissues, or allergic skin conditions, such as eczema. Additionally, diagnostic tests may be used to determine whether a subject has asthma or has symptoms related to asthma. For example, an asthma diagnosis may be performed using a lung function test to determine the amount of air breathed in and out of the lungs, and/or the rate at which air is drawn into and/or expelled from the lungs. Diagnostic tests may also include allergy testing, airway sensitivity testing, x-rays to evaluate the subject's symptoms, and the like.

### Methods

The present disclosure provides methods for determining a likelihood of a beneficial response to statin therapy in an asthma patient. In general, the subject methods involve determining the genotype of an HLA-G allele in a sample from a patient and using the genotype information to determine a likelihood that the subject will have a beneficial response to a statin therapy for the treatment of asthma. The subject methods can optionally involve determining a statin dosage regimen, i.e., a dosage amount and frequency of administration, to be used when treating an asthma patient with a statin therapy. In some embodiments, the present disclosure provides methods of treating a patient for asthma with statins, wherein the methods are based on the patient's HLA-G genotype information.

The subject methods generally involve collecting a biological sample from a subject in order to determine the subject's HLA-G genotype. The biological sample can be any sample from which genetic material can be isolated. Such biological samples include, but are not limited to , blood, hair, skin, saliva, semen, urine, fecal material, sweat, tears, buccal tissue, epithelial cells, and various other bodily tissues and/or fluids from which genetic material can be isolated for analysis.

Embodiments of the subject methods may include treating a biological sample to isolate the genetic material in the sample for use in an HLA-G genotype analysis. For example, biological samples in accordance with the subject methods may be processed using standard procedures for isolating genetic material, such as standard procedures for isolating genetic material from tissue biopsy samples, biological fluid samples, and the like. In some embodiments, a biological sample may be collected from an individual and tested immediately. In some embodiments, a biological sample may be collected and stored under suitable conditions for later analysis, e.g., frozen, refrigerated, and/or preserved.

### Analysis of HLA-G Genotype

The subject methods generally involve determining the genotype of an HLA-G gene in a biological sample from the subject. Genotype analysis is used to determine whether the subject is homozygous (having the same nucleotide on each allele) or heterozygous (having a different nucleotide on each allele) for guanine or cytosine at position +3142 of the HLA-G gene, measured relative to the transcription start site of the HLA-G gene.

Subjects that are homozygous for a guanine at position +3142 of the HLA-G gene have a higher likelihood of responding in a beneficial manner to treatment of asthma using statins. Without being held to theory, this higher likelihood of having a beneficial response to statin therapy is due to the fact that HLA-G genes having a guanine at position +3142 are more able to interact with mirs 148a, 148b, and 152, which results in down-regulation of the expression of HLA-G and leads to a reduction in asthma symptoms.

Subjects that have a cytosine at position +3142 of one or both HLA-G alleles have a lower likelihood of responding in a beneficial manner to asthma treatment using statins. Without being held to theory, this lower likelihood of having a beneficial response to statin therapy is due to the fact that HLA-G alleles having a cytosine at position +3142 are less able to interact with mirs 148a, 148b, and 152, which results in normal expression of HLA-G and no reduction in asthma symptoms.

In some embodiments, the subject methods provide for the determination of a likelihood that a patient will have a beneficial response to statin therapy for the treatment of asthma based on the patient's HLA-G genotype information. If the patient is homozygous for guanine at position +3142 of the HLA-G gene, then the patient has a higher likelihood of having a beneficial response to statin therapy for the treatment of asthma. If the patient has a cytosine at position +3142 of one or both of his/her HLA-G alleles, then the patient has a lower likelihood of having a beneficial response to statin therapy for the treatment of asthma.

### Selection of Statin Dose and/or Dosage Regimen

In some embodiments, the subject methods are used to facilitate selection of a statin dose and/or dosage regimen based on a patient's HLA-G genotype information and based on the particular statin selected for treatment.

After evaluating the patient's response to the statin administered for treatment of asthma in accordance with the methods of the present disclosure, the dose and/or dosage regimen may be adjusted as needed to achieve a desired clinical outcome. Following the initial treatment, for example, the statin dose administered to the patient can be adjusted as needed (i.e., reduced or increased in at least one of dose or frequency) based on any continuation and/or change in severity of the patient's asthma symptoms. Accordingly, the subject methods generally involve titrating the statin dose administered to a patient to treat the patient for asthma in order to achieve a desired clinical outcome.

The methods of the present disclosure relating to determining a subject's HLA-G genotype can serve as an aid to the clinician in selecting a statin for use in asthma therapy based on the likelihood of response based on his/her HLA-G genotype, selection of an initial dose or dosage regimen for the subject based on his/her HLA-G genotype, as well as guidance as to the likelihood of the need for modification and type of modification of the dose or dosage regimen based on his/her HLA-G genotype (e.g., increase or decrease in dose and/or frequency).

Initial doses can be based upon recommended doses for treatment of hypercholesterolemia. Table 1, below, provides examples of statins that may be used in connection with the subject methods, along with the recommended starting dose and overall dose range for each statin in the context of treatment of hypercholesterolemia.

TABLE 1
Comparison of statin starting dosage and overall dosage ranges.

| Name of Statin | Starting Dose | Dose Range |
|---|---|---|
| Fluvastatin | about 40 mg/day | 20-80 mg/day |
| Lovastatin | about 40 mg/day | 10-80 mg/day |
| Pravastatin | about 40 mg/day | 10-80 mg/day |
| Rosuvastatin | about 10 mg/day | 5-40 mg/day |
| Simvastatin | about 20-40 mg/day | 10-80 mg/day |
| Pitavastatin | about 2 mg/day | 1-4 mg/day |

In some embodiments, the subject methods may be used to guide a clinician as to whether statins are an appropriate therapeutic option for an asthma patient. For example, patients that are homozygous for cytosine at position +3142 of the HLA-G gene are unlikely to have a beneficial response to statin therapy for the treatment of asthma, and other treatment options may therefore be more appropriate for such patients.

In some embodiments, a patient's HLA-G genotype information can be used to guide the clinician in selecting an initial statin dose to administer to the patient. For example, patients that are homozygous for guanine at position +3142 of the HLA-G gene have a higher likelihood of having a beneficial response to statin therapy for the treatment of asthma. Accordingly, such patients may benefit from a statin dose that is the same or lower than the recommended initial statin dose for purposes of treating hypercholesterolemia, and may be lower than the lowest recommended dose or dosage regimen for a statin for purposes of treating hypercholesterolemia (e.g., by increasing the duration between doses in a dosage regimen, e.g., rather than once daily, every other day, etc.). Based on the patient's HLA-G genotype information, the clinician may therefore select the same or a lower initial statin dose and/or dosage regimen for guanine homozygous patients in order to treat them for asthma.

In some embodiments, a patient's HLA-G genotype information can be used to guide the clinicians approach to modifying a statin dosage regimen that is administered to the patient for the treatment of asthma. For example, in some embodiments, the direction of a change in statin dose (i.e., an increase or a reduction) may be selected by the clinician based on the patient's HLA-G genotype information. In some embodiments, for example, a patient is given an initial recommended dose for a particular statin and, based on any observed changes in the patient's asthma symptoms, the statin dose is appropriately titrated to achieve a desired clinical outcome using the lowest possible dosage regimen.

In some embodiments, for example, following administration of the recommended starting dose of a statin, if the patient's symptoms decrease in severity, the dose may be reduced to determine whether the reduction in the severity of symptoms can be maintained using a lower dose. In some embodiments, following administration of the recommended starting dose, if the patient's symptoms decrease in severity, the dose may be increased to see whether a further reduction in the severity of symptoms can be achieved. Patient's having one or more HLA-G alleles that have a guanine at position +3142 are more likely to be able to achieve and maintain a reduction in asthma symptoms due to statin therapy.

Conversely, if a patient's symptoms do not decrease in severity following administration of the recommended starting dose of a statin, the dose may be increased. Patient's having one or more HLA-G alleles that have a cytosine at position +3142 are more likely to require an increase in statin dose in order to achieve a reduction in the severity of their asthma symptoms, as these patients are less likely to have a beneficial response to statin therapy for the treatment of asthma.

### Genotyping Techniques

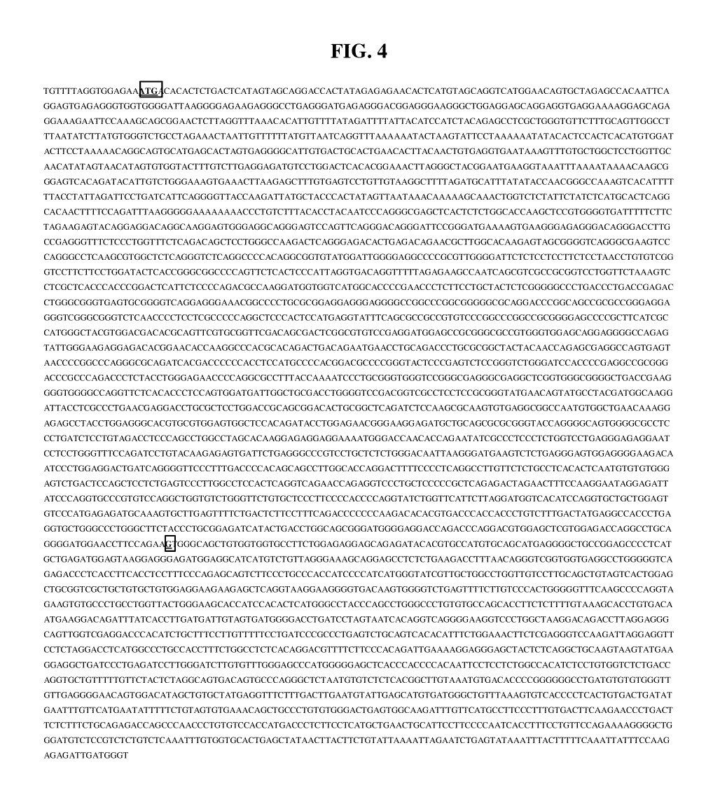

Genotyping techniques according to embodiments of the present disclosure generally involve determining the identity of a nucleotide at a particular position in the 3′ untranslated region (UTR) of the HLA-G gene of a subject. In some embodiments, genotyping analysis may be carried out by amplifying a particular target region and sequencing the amplification product to determine the identity of a nucleotide at a particular position of interest using, e.g., standard sequencing techniques. In some embodiments, a target region may be directly sequenced using, e.g., standard sequencing techniques. The human HLA-G gene can be found in the HLA region on the short arm of chromosome 6 (6p21.3). Examples of HLA-G sequences include, e.g., GenBank Accession No. JQ013009.1, and naturally-occurring allelic variants thereof. The sequence of GenBank Accession No. JQ013009.1 is provided in FIG. 4, and serves as a reference sequence for purposes of describing the methods of the present disclosure. Other examplary HLA-G gene sequences include Genbank Accession Number NG_{—}029039.

A single nucleotide polymorphism (SNP) is a DNA sequence variation occurring when a single nucleotide in the genome differs between members of a species or between paired chromosomes in an individual. Variation in the nucleotide sequence may include insertions, deletions, or substitutions. These variations result in two or more alleles, depending on the number of polymorphisms in a specific locus. These alleles then form the basis for a genotype of an individual member of the species. SNPs may fall within coding sequences of genes, noncoding regions of genes, or in intergenic regions between genes.

The methods of the present disclosure generally involve determining a genotype of a SNP in the 3′ untranslated region (UTR) of the HLA-G gene positioned at +3142 with respect to the transcription start site, and which is either guanine (G) or cytosine (C). This SNP is referred to as “rs1063320”, “SNP 3127”, or “HLA-G+3142 G/C”, which terms are used interchangeably herein. The HLA-G+3124 G/C SNP is described in, e.g., U.S. Patent Application Publication No. 2009/0156532, and is located in the 3′ UTR of the HLA-G gene. An example of a flanking sequence for the HLA-G+3142 G/C SNP is:

tccgtctctgtctcaaatttgtggt (C/G) cactgagctataactta
cttctgta

where the nucleotide corresponding to position +3142 relative to the transcription start site of the HLA-G gene is indicated in bold and underlined as C/G. Any of a variety of suitable genotyping techniques may be used to determine the identity of the nucleotide that corresponds to the position indicated by the bolded and underlined C/G in the flanking sequence provided above. For reference purposes, the A nucleotide in the ATG transcription start codon of the full length HLA-G sequence is referred to as position zero. In FIG. 4, the ATG start codon is identified with a box, and the nucleotide at position +3142 is identified with a box as well.

The HLA-G gene of a given subject may include natural variations in sequence. Accordingly, the sequences provided herein are merely examples, and in no way limit the embodiments of the present disclosure. To the contrary, any of a variety of suitable genotyping techniques may be employed in the subject methods to determine the identity of a nucleotide at a position of interest within a target nucleic acid.

In some embodiments, a 365 base pair target region of the HLA-G 3′ UTR may be used for genotyping analysis. In such embodiments, suitable primers are used for amplification of the target region, and the amplification product is then sequenced to determine the identity of a nucleotide at a position corresponding to the position that is +3142 nucleotides downstream of the transcription start site of the HLA-G gene. In one embodiment, the following primers are used to amplify a 365 base pair region of the 3′ UTR of a subject's HLA-G gene:

Forward primer: CTAGAAGCTTTGTGAAACAGCTGCCC
Reverse primer: CGCTAGTCTAGATGTCTCTCAAATTTC

Following amplification, the amplification product is sequenced and the identity of the nucleotide at the position that corresponds to position +3142 in the full length HLA-G gene is determined using standard sequencing and/or alignment techniques.

HLA-G genotyping may be performed using any suitable sample containing genetic material from a subject. Genotyping can be accomplished using any suitable technique, including but not limited to restriction fragment length polymorphism analysis, polymerase chain reaction (PCR)-based analysis, DNA hybridization techniques, DNA sequencing, RNA sequencing, allele-specific oligonucleotide probe analysis, DNA arrays, and the like.

### Use of Surrogate SNPs

In some embodiments, determining the genotype of the SNP rs1063320 of the HLA-G gene can be accomplished by determining the genotype of one or more surrogate SNPs of rs1063320. A “surrogate SNP” as used herein refers to a SNP having a linkage disequilibrium (LD) percent correlation score (r²) of at least 85% or greater with respect to a SNP rs1063320 (the “reference SNP”), and includes surrogate SNPs having an LD percent correlation score of at least 90% or greater, at least 95% or greater with respect to SNP rs1063320. Such surrogate SNPs thus serve as accurate indicators of the genotype at position +3142 of the patient's HLA-G gene.

Examples of surrogate SNPs are provided in Table 2, below. Included in Table 2 is the name of each surrogate SNP, the linkage disequilibrium (LD) percent correlation score (r²) of the SNP with respect to SNP rs1063320, and an example of a flanking sequence surrounding the SNP. Three of these SNPs, rs1610678, rs915669 and rs915668 are in perfect LD with rs1063320 in Yoruban (YRI) and a second European (TSI) population. The fact that these SNPs are in perfect LD in both Caucasian and African population indicates that these SNPs are in very tight LD in all populations.

TABLE 2
Examples of Surrogate SNPs.

| | r² with | | | |
|---|---|---|---|---|
| | rs1063320 | rs1063320 | | |
| | in CEU | surrogate | | |
| SNP | LD | C allele | G allele | Flanking Sequence |
| rs1610678 | 100 | G | A | GCTCGAATTGAGAAACTAGTTTTTTC[A/G]ATAACTGGCTATTCGATCAGCATTC |
| rs915668 | 100 | C | G | ACAGCCCATCACATGCTCAATACATT[C/G]AAGTCAAAGAAACCTCATAGCACAG |
| rs915669 | 100 | A | C | AGAAACCTCATAGCACAGCTATGTCC[A/C]CTGTTCCCCTCAACAACCCACACAC |
| rs1611165 | >90 | C | T | TACAACTTCTAACTGATAGAGTAATG[C/T]TGACATAACAGTTTGTGACTCTGGG |
| rs1632942 | >90 | G | A | TCCTCTCCTTGTGCTAGGCCAGGCTG[A/G]GAGGTCTACAGGAGATCAGGGAGGC |
| rs1736920 | >90 | C | T | GGGGTCTGAAAGGAAGAGTCAGAAAA[C/T]TCAAGCACTTTGCATCTCTCATGGG |
| rs1736927 | >90 | G | T | GCATGGAGGTGGGGGTCGTGATCTGC[G/T]CCCTGGGCCGGAGTTACTCACTGGC |
| rs1610677 | 90 | G | A | TTTTTTCAATAACTGGCTATTCGATC[A/G]GCATTCTATAATACACTTAACATCT |
| rs1632933 | 90 | A | G | GGACCTTCTCCTGACCTGTGATTACT[A/G]GGATCAGGTCCCCATCACTACAATC |
| rs1632941 | 90 | G | A | CAGGACCAGAGGGAGGGCGATATTCT[A/G]GTGTTGGTCCCATTTTCCTCCTCTC |
| rs1736936 | 90 | T | C | ATCCCCACCACCCTCTCACTCCTGAA[C/T]TGTGGCTCTAGCACTGTTCCATGAC |

Thus for example, detection of a guanine nucleotide in surrogate SNP rs1610678 indicates that the nucleotide in SNP rs1063320 is a cytosine. Further, detection of an adenine nucleotide in surrogate SNP rs1610678 indicates that the nucleotide in SNP rs1063320 is a guanine

### Detection Using PCR-Based Methods

The subject methods generally provide for the determination of a subject's HLA-G genotype using PCR-based methods. PCR-based methods involve the use of primers that amplify a target region of interest. If the target region is not present, amplification of the nucleic acid will not take place. In this regard, PCR-based methods may be used to detect whether a subject has a particular HLA-G genotype. The use of PCR is described in a variety of publications, including, e.g., “PCR Protocols (Methods in Molecular Biology)” (2000) J. M. S. Bartlett and D. Stirling, eds, Humana Press; and “PCR Applications: Protocols for Functional Genomics” (1999) Innis, Gelfand, and Sninsky, eds., Academic Press. Such PCR-based methods may involve isolating nucleic acid from a sample, contacting the nucleic acid with one or more primers that specifically hybridize with a nucleic acid encoding an HLA-G sequence under conditions such that hybridization and amplification of the template nucleic acid molecules in the sample occurs, and detecting the presence, absence, and/or relative amount of an amplification product that can be compared to that of a control sample.

Detection of an amplification product of the expected size will be an indication that an HLA-G sequence of interest is present in the test nucleic acid sample. Parameters such as hybridization conditions and primer length, and position of the HLA-G sequence of interest may be chosen such that hybridization and/or primer extension will not occur unless a nucleic acid having a sequence complementary to that of the primer(s) is also present in the sample nucleic acid. Those of ordinary skill in the art are well aware of how to select and vary such parameters. See, e.g., Saiki et al. (1986) Nature 324:163; and Saiki et al (1989) Proc. Natl. Acad. Sci. USA 86:6230. The primers can further be designed such that the length of the amplification products will be indicative of the HLA-G genotype.

A detectable label may be included in an amplification reaction. Suitable labels include fluorochromes, e.g. fluorescein isothiocyanate (FITC), rhodamine, Texas Red, phycoerythrin, allophycocyanin, 6 carboxyfluorescein (6 FAM), 2′,7′ dimethoxy 4′,5′ dichloro 6 carboxyfluorescein (JOE), 6 carboxy X rhodamine (ROX), 6 carboxy 2′,4′,7′,4,7 hexachlorofluorescein (HEX), 5 carboxyfluorescein (5 FAM) or N,N,N′,N′ tetramethyl 6 carboxyrhodamine (TAMRA), radioactive labels, e.g. 32P, 35S, 3H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification may be labeled, so as to incorporate the label into the amplification product.

In some embodiments, the HLA-G genotype is detected using any of a variety of real-time PCR methods, which methods allow for detection of PCR products created during the exponential phase of amplification to facilitate determination of the amount of the amplified transcript present in the sample. In general, real-time PCR (RT-PCR) involves use of amplification primers, and a detectably labeled probe. For example, in the present methods, the probe can be designed to specifically hybridize to a target region of an HLA-G-encoding sequence, such as the region near position +3142. Examples of RT-PCR methods are described in, for example, U.S. Pat. No. 5,210,015; and U.S. Pat. No. 6,548,250.

In some embodiments, the invention features a probe or a set of probes suitable for use in RT-PCR, which probe(s) specifically hybridize to an HLA-G target region (e.g., the region near position +3142) and can be used to determine whether a subject is homozygous for a G or a C at position +3142 of the HLA-G gene, or whether a subject is heterozygous (having one C and one G nucleotide) at position +3142 of the HLA-G gene. Such probes generally include both a fluorophore and a quenching agent attached to the probe. When the probe is intact, the fluorescence of the fluorophore is quenched by the quencher. If the probe specifically hybridizes to a target region, the probe is cleaved between the fluorophore and the quencher, allowing full emission of the fluorophore fluorescence. Quenching involves transfer of energy between the fluorophore and the quencher, the emission spectrum of the fluorophore and the absorption spectrum of the quencher overlap (e.g., as in where the fluorophore is rhodamine 590 and the quencher is crystal violet). In a related embodiment, a probe is cleaved when amplification from a primer positioned 5′ of the probe occurs. Further embodiments of this aspect of the invention are described in, e.g., U.S. Pat. No. 5,210,015; and U.S. Pat. No. 6,548,250.

In some embodiments, the subject methods include direct DNA sequencing, wherein amplification and sequence determination are performed in one single reaction.

### Hybridization Analysis

Hybridization with a target sequence may also be used in HLA-G genotyping analysis. Hybridization analysis can be carried out in a number of different ways; including, but not limited to: Southern blots, dot blots, microarrays, etc. The hybridization pattern of a control and variant sequence to an array of oligonucleotide probes immobilized on a solid support, as described in U.S. Pat. No. 5,445,934, or in WO 95/35505, may also be used as a means of determining HLA-G genotype. For instance, identification of a polymorphism in a nucleic acid sample can be performed by hybridizing both sample and control nucleic acids to high density arrays containing hundreds or thousands of oligonucleotide probes. Cronin et al. (1996) Human Mutation 7:244-255; and Kozal et al. (1996) Nature Med. 2:753-759.

Hybridization reactions can be performed under conditions of different stringency. Conditions that increase stringency of a hybridization reaction are widely known and published in the art. See, e.g., Sambrook et al. (1989). Examples of relevant conditions include (in order of increasing stringency): incubation temperatures of 25° C., 37° C., 50° C. and 68° C.; buffer concentrations of 10×SSC, 6×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or deionized water. Examples of stringent conditions are hybridization and washing at 50° C. or higher and in 0.1×SSC (9 mM NaCl/0.9 mM sodium citrate).

Stringent conditions for DNA/DNA hybridization are as described by Sambrook et al. Molecular Cloning, A Laboratory Manual, 2nd Ed., Cold Spring Harbor Laboratory Press, Cold Spring Harbor, N.Y., 1989, herein incorporated by reference. For example, see page 7.52 of Sambrook et al. Alternatively, various methods are known in the art that utilize oligonucleotide ligation as a means of detecting polymorphisms. See, e.g., Riley et al. (1990) Nucleic Acids Res. 18:2887-2890; and Delahunty et al. (1996) Am. J. Hum. Genet. 58:1239-1246.

The hybridizaton probes may be coupled to labels for detection. As with primers, several methods and compositions for derivitizing oligonucleotides with reactive functionalities that permit the addition of a label are known in the art. For example, several approaches are available for biotinylating probes so that radioactive, fluorescent, chemiluminescent, enzymatic, or electron dense labels can be attached via avidin. See, e.g., Broken et al., Nucl. Acids Res. (1978) 5:363-384 which discloses the use of ferritin-avidin-biotin labels; and Chollet et al. Nucl. Acids Res. (1985) 13:1529-1541 which discloses biotinylation of the 5′ termini of oligonucleotides via an aminoalkylphosphoramide linker arm. Several methods are also available for synthesizing amino-derivatized oligonucleotides which are readily labeled by fluorescent or other types of compounds derivatized by amino-reactive groups, such as isothiocyanate, N-hydroxysuccinimide, or the like, see, e.g., Connolly (1987) Nucl. Acids Res. 15:3131-3139, Gibson et al. (1987) Nucl. Acids Res. 15:6455-6467 and U.S. Pat. No. 4,605,735 to Miyoshi et al. Methods are also available for synthesizing sulfhydryl-derivatized oligonucleotides, which can be reacted with thiol-specific labels, see, e.g., U.S. Pat. No. 4,757,141 to Fung et al., Connolly et al. (1985) Nuc. Acids Res. 13:4485-4502 and Spoat et al. (1987) Nucl. Acids Res. 15:4837-4848. A comprehensive review of methodologies for labeling DNA fragments is provided in Matthews et al., Anal. Biochem. (1988) 169:1-25.

For example, probes may be fluorescently labeled by linking a fluorescent molecule to the non-ligating terminus of the probe. Guidance for selecting appropriate fluorescent labels can be found in Smith et al., Meth. Enzymol. (1987) 155:260-301; Karger et al., Nucl. Acids Res. (1991) 19:4955-4962; Haugland (1989) Handbook of Fluorescent Probes and Research Chemicals (Molecular Probes, Inc., Eugene, Oreg.). Preferred fluorescent labels include fluorescein and derivatives thereof, such as disclosed in U.S. Pat. No. 4,318,846 and Lee et al., Cytometry (1989) 10:151-164, and 6-FAM, JOE, TAMRA, ROX, HEX-1, HEX-2, ZOE, TET-1 or NAN-2, and the like.

Additionally, probes can be labeled with an acridinium ester (AE). Current technologies allow the AE label to be placed at any location within the probe. See, e.g., Nelson et al. (1995) “Detection of Acridinium Esters by Chemiluminescence” in Nonisotopic Probing, Blotting and Sequencing, Kricka L. J. (ed) Academic Press, San Diego, Calif.; Nelson et al. (1994) “Application of the Hybridization Protection Assay (HPA) to PCR” in The Polymerase Chain Reaction, Mullis et al. (eds.) Birkhauser, Boston, Mass.; Weeks et al., Clin. Chem. (1983) 29:1474-1479; Berry et al., Clin. Chem. (1988) 34:2087-2090. An AE molecule can be directly attached to the probe using non-nucleotide-based linker arm chemistry that allows placement of the label at any location within the probe. See, e.g., U.S. Pat. Nos. 5,585,481 and 5,185,439.

In some embodiments, hybridization of the target nucleic acid (e.g., encoding a specific HLA-G sequence) is accomplished using a support, usually a solid support, having probe bound to a surface, e.g., to capture amplicons of target nucleic acid by hybridization of the target to the probe. Examples of preferred types of supports for immobilization of the probe include controlled pore glass, glass plates, polystyrene, avidin-coated polystyrene beads, cellulose, nylon, acrylamide gel and activated dextran.

Where a support is used, the probe may be attached to the support in a variety of manners. For example, the probe may be attached to the support through a 3′ or 5′ terminal nucleotide of the probe. In some embodiments, the probe is attached to the support by a linker that serves to distance the probe from the support surface. The linker is usually at least 15-30 atoms in length, more preferably at least 15-50 atoms in length. The required length of the linker will depend on the particular solid support used. For example, a six atom linker is generally sufficient when highly cross-linked polystyrene is used as the solid support.

A wide variety of linkers are known in the art which may be used to attach the probe to the support. The linker may be formed of any compound which does not significantly interfere with the hybridization of the target sequence to the probe attached to the support. The linker may be formed of a homopolymeric oligonucleotide which can be readily added on to the linker by automated synthesis. Alternatively, polymers such as functionalized polyethylene glycol can be used as the linker. Such polymers are preferred over homopolymeric oligonucleotides because they do not significantly interfere with the hybridization of probe to the target oligonucleotide. Polyethylene glycol is particularly preferred. The linkages between the support, the linker and the probe are normally not cleaved during removal of base protecting groups under basic conditions at high temperature. Examples of preferred linkages include carbamate and amide linkages.

### Manual and Computer-Assisted Methods and Products

The HLA-G genotype information described above can be calculated and stored manually, e.g., on a patient record. Alternatively, the above-described steps can be completely or partially performed by a computer program product. The present disclosure thus provides a computer program product including a computer readable storage medium having a computer program stored on it. The program can, when read by a computer, execute relevant calculations based on values obtained from analysis of one or more biological samples from an individual (e.g., HLA-G genotype information), convert values from genotype information into a prediction regarding the likelihood of a beneficial response to statin therapy, and guidance regarding the dosage and frequency of administration of a statin that should be administered to a subject for asthma therapy. The computer program product has stored therein a computer program for performing such calculations and providing such guidance.

### Systems and Apparatus

In some embodiments, the present disclosure provides a system for executing the program described above, which system generally includes: a) a central computing environment; b) an input device, operatively connected to the computing environment, to receive assay data, wherein the assay data can include, for example, data from assays to determine HLA-G genotype from an assay using a biological sample from the patient as described in detail above; c) an output device, connected to the computing environment, to provide information to a user (e.g., medical personnel); and d) at least one algorithm executed by the central computing environment (e.g., a processor), where the algorithm is executed based on the data received by the input device, wherein the algorithm can provide for calculation of i) a likelihood that an asthma patient will have a beneficial response to statin treatment, and, optionally, ii) a proposed statin dosage regimen, including a dosage amount and frequency of administration, to be administered to the patient for the treatment of asthma.

The present disclosure further provides a portable apparatus having a computer readable medium (e.g., a processor) that stores data, calculates values using algorithm described above, and provides an assessment of the likelihood of a beneficial response to statin therapy based on the calculation. In some embodiments, a subject apparatus (e.g., a portable apparatus) comprises: a) a device for receiving and storing patient data as described above, including assay values, calculation results, and information relating to the patient's HLA-G genotype, patient information, and the like; b) a data output device; and c) at least one algorithm stored within the computer program product within the apparatus, which algorithm, for example, assesses a prognosis or diagnosis of responsiveness to statin therapy as described above, which information is transmitted to the data output device, where the output device displays the information (e.g., “highly statin responsive,” “intermediate statin responsive,” “diminished statin responsive,”, and the like) as well as proposed or suggested dosage regimen information, to a user.

The data input device (also referred to as an operator input device) may be, e.g., a keyboard or keypad, a mouse, and the like. The processor has access to a memory, which may be any suitable device in which the processor can store and retrieve data, such as magnetic, optical, or solid state storage devices (including magnetic or optical disks or tape or RAM, or any other suitable device). The processor can include a general purpose digital microprocessor (such as is typically used in a programmable computer) suitably programmed to execute an algorithm as described above, or any hardware or software combination which will perform the required functions.

In some embodiments, the portable apparatus comprises: a) a device for determining an HLA-G genotype from a biological sample; b) a device for communicating (e.g., transmitting) the determined value to the receiving and storage device; c) a data output device; and d) an algorithm stored within a computer program product within the apparatus, which algorithm is executed to, for example, determine a likelihood that the patient will have a beneficial response to statin therapy for the treatment of asthma, as well as provide dosage regimen guidance to the user. The result is transmitted to the data output device, where the output device displays the assessment to a user, which assessment can include a prognosis/diagnosis of the likelihood of a beneficial response to statin therapy for the treatment of asthma, and optionally include a proposed dosage regimen, including a statin dosage and frequency of administration.

In general, a subject apparatus will include a computer readable medium including the programming described above. The computer program can be recorded on computer readable media, e.g., any medium that can be read and accessed directly or indirectly by a computer. Such media include, but are not limited to: magnetic tape; optical storage such as compact disc-read only memory (CD-ROM) and digital versatile disk (DVD); electrical storage media such as random access memory (RAM) and read-only memory (ROM); and hybrids of these categories such as magnetic/optical storage media. One of skill in the art can readily appreciate how any suitable computer readable media can be used to create a manufacture that includes a recording of the present programming/algorithms for carrying out the above-described methodology. In certain embodiments, the programming is further characterized in that it provides a user interface, where the user interface presents to a user the option of selecting among one or more different, including multiple different, criteria, e.g., age of individual, etc. The instructions may include installation or setup directions. The instructions may include directions for use of the apparatus.

In addition, a subject apparatus will typically include instructions for using the apparatus to carry out the subject methods. The instructions of the above-described apparatus are generally recorded on a suitable recording medium. For example, the instructions may be printed on a substrate, such as paper or plastic, etc. As such, the instructions may be present in the apparatus as a package insert, or components thereof (i.e., associated with the packaging or sub packaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the apparatus, but means for obtaining the instructions from a remote source, e.g., via the Internet, are provided. An example of this embodiment is an apparatus that includes a web address where the instructions can be viewed and/or from which the instructions can be downloaded. Conversely, means may be provided for obtaining the subject programming from a remote source, such as by providing a web address. Still further, the apparatus may be one in which both the instructions and software are obtained or downloaded from a remote source, as in the Internet or World Wide Web. Some form of access security or identification protocol may be used to limit access to those entitled to use the subject invention. As with the instructions, the means for obtaining the instructions and/or programming is generally recorded on a suitable recording medium.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Materials and Methods

The following materials and methods were used to carry out the examples discussed below:

### Cell Culture

Lymphocytes were isolated from subjects of the Cholesterol and Pharmacogenetics (CAP) clinical trial using IsoPrep™ and immortalized by EBV transformation. Simvastatin was converted to the active form with NaOH. Immortalized lymphoblastoid cell lines (LCLs) were grown at 37° C., 5% CO₂ in RPMI Medium 1640 supplemented with 10% fetal bovine serum, 500 units/ml Penicillin/Streptomycin and 2 nM GlutaMAX. HepG2 cells were obtained from ATCC and cultured under standard conditions: 37° C., 5% CO₂ in MEM supplemented with 10% fetal bovine serum, 1% sodium pyruvate and 1% nonessential amino acids. Both cell types were exposed in replicate to either 2.0 μM activated simvastatin or sham buffer for 24 hours.

Identification of microRNAs

Total RNA was extracted from statin and sham incubated HepG2 (n=1) and immortalized lymphoblastoid cell lines (n=3) using the Mir-Vana total RNA isolation kit (Ambion). RNA quantity was verified by Q-bit, and integrity verified using the Agilent Bioanalyzer. Only samples with RIN values greater than 8 were used. Small RNA sequencing libraries were prepared using the Small RNA library preparation kit (Illumina) following the manufacturer's protocol and libraries were sequenced on the Illumina GAII to a depth of 50 million reads per library. Sequences were compared to all known microRNAs in mirBASE, and statin and sham libraries compared to rank order candidate microRNAs changing in response to statin treatment.

mRNA and microRNA Quantitation

HLA-G gene expression was measured in paired RNA samples from statin and sham-treated LCLs, selected based on RNA quality and quantity, were amplified and biotin labeled using the Illumina TotalPrep-96 RNA amplification kit, hybridized to Illumina HumanRef-8v3 bead arrays (Illumina), and scanned using an Illumina BeadXpress reader. Data were read into GenomeStudio and samples were selected for inclusion based on quality control criteria: (1) signal to noise ratio (95th:5th percentiles), (2) matched gender between sample and data, and (3) average correlation of expression profiles within three standard deviations of the within-group mean (r=0.99±0.0093 for control-exposed and r=0.98±0.0071 for simvastatin-exposed bead arrays). Genes were annotated through biomaRt from ensMBL. Mir-148a, 148b and 152 were quantified with three TaqMan assays purchased from Applied Biosystems following the manufacturer's protocol. Each real time PCR reaction was performed in triplicate on an ABI PRISM 7900 Sequence Detection System with standard reagents and 125 ng cDNA.

rs1063320 Genotyping

Donors of the LCLs used in this study were genotyped on either the Illumina HumanHap300 bead chip and/or the Illumina HumanHap610 Quad bead chip. rs1063320 genotype was obtained by imputation using the software IMPUTE23 with an integrated reference panel that included 120 CEU haplotypes from the 1000 Genomes Pilot Project (1000G) and 1910 worldwide haplotypes from the HapMap Phase 3 Project (HM3).

### HLA-G Protein Quantitation

Soluble HLA-G was quantified in LCLs after simvastatin or sham incubation as described above using the soluble HLA-G BioAssay ELISA. All measurements were performed in triplicate.

### Sequences of MicroRNAs

The sequences of the mirs are as follows:
mir-148a; Accession number: MIMAT0000243 (accession number corresponds to the miRBase, http://microma.sanger.ac.uk). The sequence for mir-148a is UCAGUGCACUACAGAACUUUGU.
mir-148b; Accession number: MIMAT0000759 (accession number corresponds to the miRBase, http://microma.sanger.ac.uk). The sequence for mir-148b is UCAGUGCAUCACAGAACUUUGU.
mir-152; Accession number: MIMAT0000438 (accession number corresponds to the miRBase, http://microma.sanger.ac.uk). The sequence for mir-152 is UCAGUGCAUGACAGAACUUGGG.

### Example 1

### Mirs are Statin Responsive

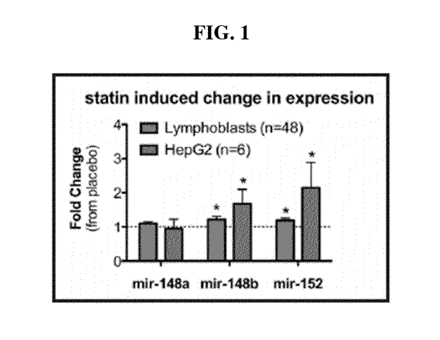

To determine if mir-148a, 148b or 152 transcript levels are statin responsive, CAP LCLs (n=48) and HepG2 cell lines (n=6) were incubated with either 2.0 pM simvastatin or sham buffer for 24 hours, and subsequent changes in transcript levels were quantified using quantitative real-time PCR. Mir-148b and 152 were up-regulated with statin treatment in both cell types (p<0.05) (FIG. 1).

The results indicate that the mirs correlate with HLA-G expression levels, and their effect is dictated in part by the identity of the nucleotide present at position +3142 of the HLA-G gene. When a guanine is present at position +3142, the mirs are associated with reduced expression of HLA-G, which leads to a reduction in asthma symptoms. When a cytosine is present at position +3142, the mirs have no detectable effect on HLA-G expression, and thus no detectable effect on asthma symptoms. The mirs are referred to as mir-148a, mir-148b, and mir-152, and are described in, e.g., U.S. Patent Application Publication No. 2009/0156532.

### Example 2

### Mir Levels and HLA-G Levels are Correlated Based on Genotype

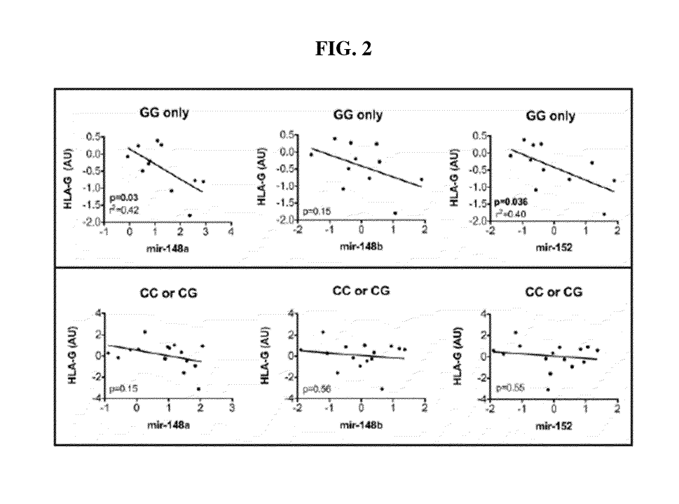

FIG. 2 shows that higher HLA-G transcript levels are correlated with lower mir-148a and mir-152 expression in LCLs derived from rs1063320 GG homozygotes (p<0.05, n=11). In contrast, there were much weaker and non-significant relationships between HLA-G mRNA and mir-148a, 148b and 152 in LCLs from either CC or CG rs1063320 donors (n=15).

### Example 3

### HLA-G mRNA and Protein Levels Differ Based on Genotype

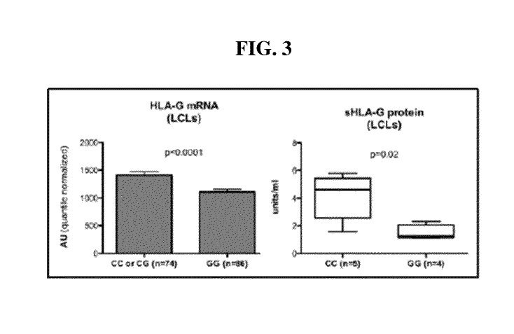

Under basal growth conditions, CAP LCLs homozygous for the rs1063320 “G” allele had reduced HLA-G transcript and protein levels compared to cells with at least one copy of the “C” allele (p<0.05) (FIG. 3). These results support the utility of this genotype model.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for determining a likelihood of a beneficial response to statin therapy in an asthma patient, comprising:
genotyping a single nucleotide polymorphism (SNP) rs1063320, or a surrogate SNP thereof, in a sample of genetic material from the patient, wherein said genotyping determines if the nucleotide of the SNP rs1063320 of one or both alleles of the patient's HLA-G gene is a guanine or a cytosine;
wherein a cytosine at position +3142 of one or both alleles is negatively associated with the likelihood that the patient will have a beneficial response to statin treatment; and
wherein a guanine at position +3142 of one or both alleles is positively associated with the likelihood that the patient will have a beneficial response to statin treatment.

2. The method of claim 1, comprising:
generating a report indicating the genotype at position +3142 of one or both of the HLA-G alleles in the asthma patient.

3. The method of claim 2, wherein the report includes an indication of the likelihood of a beneficial response to statin therapy in the asthma patient based on the genotype at position +3142 of one or both of the patient's HLA-G alleles.

4. A method for determining a statin dose to administer to an asthma patient, the method comprising:
genotyping a single nucleotide polymorphism (SNP) rs1063320, or a surrogate SNP thereof, in a sample of genetic material from the patient, wherein said genotyping determines if the nucleotide of the SNP rs1063320 of one or both alleles of the patient's HLA-G gene is a guanine or a cytosine;
wherein a cytosine at position +3142 of one or both alleles indicates a higher dose of statin than is indicated for a patient with a guanine at position +3142 of both alleles.

5. The method of claim 4, comprising:
genotyping a single nucleotide polymorphism (SNP) rs1063320, or a surrogate SNP thereof, in a sample of genetic material from the patient, wherein said genotyping determines if the nucleotide of the SNP rs1063320 of one or both alleles of the patient's HLA-G gene is a guanine or a cytosine;
wherein
if a cytosine is present at position +3142 of one or both alleles, an initial recommended statin dose is administered to the patient and the statin dose is optionally increased until a reduction in asthma symptoms is observed; and
if a guanine is present at position +3142 of both alleles, an initial recommended statin dose is administered to the patient and the statin dose is optionally decreased so long as adequate control of symptoms is maintained.

6. The method of claim 5, comprising:
generating a report with information to guide a clinician's recommendation with respect to statin dose for the patient based on the nucleotide at position +3142 of one or both alleles of the patient's HLA-G gene.

7. A method of treating a patient having asthma, comprising:
determining a statin dose according to the method of claim 4; and
administering the statin dose to the patient.

8. A method of treating a patient having asthma, comprising:
administering a statin to the patient in an amount effective to treat asthmatic symptoms in the patient, wherein the patient has a guanine at position +3142 of both HLA-G alleles, or has a cytosine at position +3142 of both HLA-G alleles, or has a guanine at position +3142 of one HLA-G allele and has a cytosine at position +3142 of the other HLA-G allele.
